(19) United States
(12) Patent Application Publication
(10) Pub. No.: US 2007/0225707 A1
(43) Pub. Date: Sep. 27, 2007

(54) Orthopedic spinal devices fabricated from two or more materials

(75) Inventors: Paul Wisnewski, Maple Grove, MN (US); Chris Johnson, Germantown, TN (US); Naim Istephanous, Roseville, MN (US)

Correspondence Address: KRIEG DEVAULT LLP, ONE INDIANA SQUARE, SUITE 2800, INDIANAPOLIS, IN, 46204-2709, US

(73) Assignee: SDGI Holdings, Inc.

(21) Appl. No.: 11/386,592

(22) Filed: Mar. 22, 2006

(51) Int. Cl. A61F 2/30

(52) U.S. Cl. 606/61

(57) ABSTRACT

This invention relates to medical devices including spinal orthopedic implants and surgical instruments including a component having at least two materials providing differing performance characteristics in a unitary structure. The differing materials can be bonded or fused to one another at an atomic level to be joined to form a unitary implant or instrument component having differing materials.

### BACKGROUND

The present invention relates to medical devices formed of at least two materials to provide differing performance characteristics and to methods of implanting and employing the medical devices into patients in need of treatment.

Stabilization of adjacent bony portions can be completed with an implant positioned between the bony portions and/or an implant positioned along the bony portions. The implants can be rigid to prevent motion between the bony portions, or can be flexible to allow at least limited motion between the bony portions while providing a stabilizing effect. As used herein, bony portions can be portions of bone that are separated by one or more joints, fractures, breaks, or other space.

It can be desirable to provide a medical device having different performance characteristics to provide the desired stabilization effect or to provide desired performance characteristics. Such medical devices can be provided with multiple components to accomplish this objective. However, the fabrication of multiple components to achieve differing performance characteristics can result in inefficiencies, and can be cumbersome to assemble and apply during surgery.

Consequently, there is a continuing need for advancements in the relevant field including new implant and device designs, new material compositions, and configurations for use in medical devices that reduce the number of components of a medical device while improving or enhancing functionality. The present invention is such an advancement and provides a variety of additional benefits and advantages.

### BRIEF DESCRIPTION OF THE DRAWINGS

FIG. 1 is a sectional view of an implant assembly according to one embodiment.

FIG. 2 is a sectional view of a load transfer member of the implant assembly of FIG. 1.

FIG. 3 is an elevation view of an implant component according to another embodiment.

FIG. 4 is an elevation view of a spinal column segment with a pair of implant components of FIG. 3 secured thereto.

FIG. 5 is a cross-sectional view along another embodiment implant component.

FIG. 6 is an elevation view of another embodiment implant component.

FIG. 7 is a sectional view of a portion of the implant component of FIG. 6 with an anchor for securing the component to a bony portion.

FIG. 8 is a diagrammatic view of another embodiment medical device in the form of a surgical instrument

### SUMMARY

The present invention relates to medical devices including implant components and surgical instrument components providing an integral, unitary body comprised of at least two materials each having a different performance characteristic to enhance functionality of the device.

In one form, an orthopedic device includes an implant positionable in a patient in a surgical procedure and a bone anchor assembly for engagement with at least one bony portion of the patient. The bone anchor assembly includes a receiver engageable to the implant and a bone engaging member extending from said receiver. The bone engaging member includes a first portion to engage the bony portion and a second portion adjacent the receiver. The assembly also includes a load transfer member with a first portion adjacent the second portion of the bone engaging member and a second portion adjacent the implant. The first portion is comprised of a first material having a first performance characteristic and the second portion is comprised of a second material having a second, different performance characteristic from the first performance characteristic. The first and second materials are joined at an atomic level to provide an integral, unitary structure.

In another form, an orthopedic device includes a body including at least a first portion and a second portion. The first portion and second portion are integral and unitary with the body, and the first portion consists essentially of a first material having a first performance characteristic and the second portion consists essentially of a second material having a second performance characteristic that differs from the first performance characteristic. A system can be provided to secure the body to the spinal column.

In another form, an orthopedic device includes an elongate body positionable along bony portions. The body includes at least a first portion extending along at least a first part of a length of the body and a second portion extending along at least a second part of a length of the body. The first and second portions provide an integral, unitary structure with the body and the first portion is comprised of a first material having a first performance characteristic and the second portion is comprised of a second material having a second performance characteristic that differs from the first performance characteristic. An articulating bone screw assembly can be provided for engagement with the bony portion to secure the elongate member therealong.

In another form, an elongated spinal implant device includes a component comprising a first layer composed of a first metal material and positioned between second and third layers composed of a different, second metal material. The first metal material has a first stiffness that is less than a second stiffness of the second metal material, the first component having a length between opposite ends thereof sized to extend between and be secured to at least two adjacent vertebrae. The first, second and third layers provide an integral, unitary structure.

In another form, a method of fabricating a spinal implant includes: providing a first portion of a component composed of a first metal; providing a second portion of the component composed of a second metal, the second metal having a performance characteristic that differs from a performance characteristic of the first metal; and joining said first portion and said second portion into an integral unitary structure for the component, the component having a length sized to extend along at least first and second vertebrae when positioned along the spinal column.

In another form, a method of fabricating a spinal implant includes: providing a first portion of a component composed of a first metal; providing a second portion of the component composed of a second metal, the second metal having a performance characteristic that differs from a performance characteristic of the first metal; and joining said first portion and said second portion into an integral unitary structure for the component, the component having a seating surface formed by the first portion and an engaging surface formed by the second portion.

In another form, a surgical instrument includes a body including at least a first portion and a second portion. The first portion and second portion are integral and unitary with the body, and the first portion consists essentially of a first material having a first performance characteristic and the second portion consists essentially of a second material having a second performance characteristic that differs from the first performance characteristic. One of the first and second portions can be an end effector configured to perform a surgical procedure in the patient.

Further objects, features, aspects, forms, advantages and benefits shall become apparent from the description and drawings contained herein.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

The present invention includes implantable medical devices that are constructed, or at least partly constructed to include at least one component that includes multiple materials in an integral, unitary structure to provide differing performance characteristics for the component. In general, the component can be formed of metal and metal alloys that have been metallurgically joined at an atomic level by, for example, fusing or bonding, to provide the component with an integral, unitary structure of at least two materials having differing performance characteristics along, about or within the component.

The metal and metal alloys and their associated performance characteristics can be specifically selected and tailored for specific medical applications. The two or more materials can be selected and treated to accomplish two different goals. For example, the materials can be selected for their associated stiffness, rigidity, hardness, deformability, elasticity, flexibility, fatigue resistance, wear resistance, radiopacity or radiographic imaging properties, or load carrying capability. The two materials can then be appropriately combined to provide the implantable medical device with a unitary component that exhibits superior performance characteristics.

Specific examples of medical devices that are included within the scope of the present invention include orthopedic implants such as spinal implants that are employed alone or with other components to stabilize one or more vertebral levels. Such components can form all or a portion of the medical device, and the medical device may be an intervertebral prosthesis, intravertebral prosthesis, or extravertebral prosthesis such as a bone plate, spinal rod, rod connector, or bone anchor. The medical devices can be used to treat a wide variety of animals, particularly vertebrate animals and including humans. Also contemplated are surgical instruments where one or more portions of the instrument including a material profile having two or more metals or metal alloys is employed to perform surgical procedures. Such surgical instrument can include cutting instruments, drills, reamers, distractors to separate bone portions, forceps, rongeurs, resection instruments, endoscopes, implant inserter instruments, bone tamps, retractors, and cannulae, for example

The medical devices can be formed to include one or more components having a material profile that includes, for example, a first metal or metal alloy that is fused, diffused, or bonded for joining at an atomic level with a second metal or metal alloy. In preferred embodiments, there is no need or requirement for a bonding layer between the first and second metals or metal alloys, although the use of a bonding layer is not precluded. However, it will be understood by those skilled in the art that depending upon the method of fabrication, various zones, regions or diffusion layers may exist between the various materials comprising the component that could be considered to be a bonding layer. For the present invention, the term “bonding layer” is intended to mean that an intermediate layer, region or zone, that has materials that include at least in part both of the first and second materials comprising the component of the medical device and/or a layer of third material between the first and second materials.

The at least two metals or metal alloys can be bonded, fused, and/or diffused with one another to be joined at an atomic level to form an integral, unitary component for the medical device that has differing performance characteristics based on the properties of the particular metal or metal alloy. These devices can provide particular advantages for use in stabilization of articulating joints such as spinal implants which are used to treat spinal disorders. Additionally, the medical device can be used for stabilization of other joints such as the knee, hip, shoulder, and the like, and for stabilization between any adjacent bony portions separated by a fracture, defect, space or the like.

The materials for use in the medical devices are selected to be biologically and/or pharmacologically compatible. Further, the preferred materials exhibit minimal toxicity, either as part of the bulk device or in particulate form. The individual components in the device are also biocompatible. In particularly preferred embodiments, the metal materials include at least one material that has been accepted for use by the medical community, particularly the FDA and surgeons.

The metal and metal alloys can be selected from a wide variety of biocompatible metals and metal alloys. Specific examples of biocompatible metals and metal alloys for use include titanium and its alloys, zirconium and its alloys, niobium and its alloys, stainless steels, cobalt and its alloys, and mixtures of these materials. In particular embodiments, the metal material includes commercially pure titanium metal (CpTi) or a titanium alloy. Examples of titanium alloys for use include Ti-6Al-4V, Ti-6Al-6V, Ti-6Al-6V-2Sn, Ti-6Al-2Sn-4Zr-2Mo, Ti—V-2Fe-3Al, Ti-5Al-2.5Sn, and TiNi. These alloys are commercially available in a sufficient purity from one or more of the following vendors: ATI Allvac; Timet Industries; Specialty Metals; and Teledyne Wah Chang. In one embodiment, the materials are specifically selected to provide desired load carrying capability with a desired performance characteristics to prevent movement between one or more bony portions or a desired performance characteristic to permit at least some limited movement between adjacent bony portions.

The medical devices include one or more components that can be prepared by forming an integral, unitary structure including at least two metals or metal alloys. Preferred processes for forming the unitary components include: conventional melting technology, such as, casting directional solidification, liquid injection molding, laser sintering, laser-engineered net shaping, powder metallurgy, metal injection molding (MIM) techniques; and mechanical processes such as rolling, forging, stamping, drawing, and extrusion. Also contemplated are cladding processes that can include cladding techniques; thermal spray processes that include: wire combustion, powder combustion, plasma flame and high velocity Ox/fuel (HVOF) techniques; pressured and sintered physical vapor deposition (PVD); chemical vapor deposition (CVD); or atomic layer deposition (ALD), ion plating and chemical plating techniques.

For use in the spine, the component is fabricated to exhibit suitable strength to withstand the biomechanical stresses and clinically relevant forces without permanent deformation. For devices that are not implanted in the or around the spine, the component can be fabricated to withstand the biomechanical forces exerted by the associated musculoskeletal structures. In a particular embodiment, one portion of the component is composed of titanium, (CpTi) and transitions to a second material that has a differing performance characteristic, such as a titanium alloy of Ti-15Mo or Ti-6Al-4V. Thus, the performance characteristic of the component will vary depending on the location of the portions having the various materials. For example, a stiff or stiffer portion of the component can be employed where movement is not desired, and a less stiff portion of the component can be employed where at least some motion is desired or acceptable.

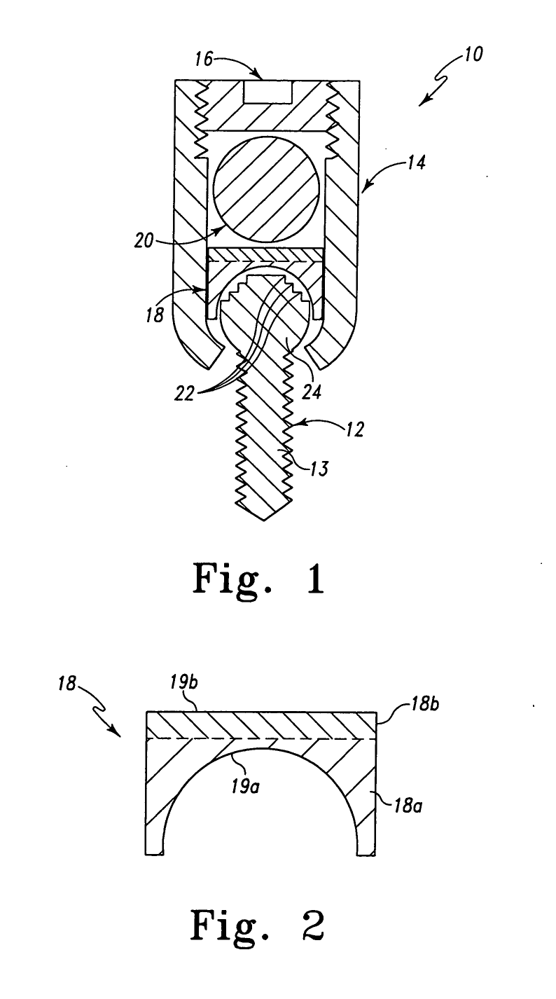

Metallic spinal implants can be fabricated so that one or more components or sub-components that include at least two constituent metals comprising different portions of the device. One specific application includes a multi-axial spinal anchor, as shown in FIG. 1. Anchor 10 includes a bone engaging member 12, a receiver 14, an engaging member 16, and a load transfer member 18. Bone engaging member 12 can be pivotally mounted, engaged, or captured in receiver 14 so that a first bone engaging portion 13 thereof can assume any one of a number of angular orientations relative to receiver 14 and/or connecting member 20. Other embodiments contemplate a uni-axial arrangement between receiver 14 and bone engaging member 12.

An elongate connecting member 20, such as a spinal rod, can be positioned in receiver 14 between load transfer member 18 and engaging member 16. Engaging member 16 can be threadingly advanced along receiver 14 to secure connecting member 20 against load transfer member 18. Other embodiments contemplate connecting member 20 can be positioned about or around receiver 14. It is also contemplated that engaging member 16 can be secured about or around receiver 14.

In the illustrated embodiment, load transfer member 18 is secured against bone engaging member 12 to secure bone engaging member 12 and connecting member 20 in position relative to one another. Bone engaging member 12 can include a head 24 with a number of ridges 22 extending thereabout. Load transfer member 18 engages the ridges 22 about head 24 or other suitable structure of bone engaging member 12 to lock bone engaging member 12 in position in receiver 14.

As further shown in FIG. 2, load transfer member 18 includes a lower portion 18a that sits on head 24 of bone engaging member 12 and an upper portion 18b that is adjacent to and in contact with connecting member 20 when it is secured with receiver 14. It is desirable for lower portion 18a to be deformable to allow or facilitate ridges 22 biting into lower portion 18a and achieve locking of bone engaging member 12. In the illustrated embodiment, lower portion 18a includes a distally oriented concavely curved recess 19a to facilitate receipt of head 24 therein and maximize contact therewith.

In the illustrated embodiment, load transfer member 18 includes lower portion 18a formed with a first material and includes a concave lower surface that generally conforms to head 24 of bone screw portion 12. Upper portion 18b is formed of a second material that is joined with the first material to provide a unitary structure for load transfer member 18.

It is further desirable that upper portion 18b be formed of a second material that is not deformable or less deformable than the material comprising lower portion 18a in order that loading may be more effectively transferred to lower portion 18a. Thus, lower portion 18a is made from a first material that has a hardness that is less than a hardness of upper portion 18b. In the illustrated embodiment, upper portion 18b forms a seating surface 19b that contacts connecting member 20. Seating surface 19b is shown as flat or planar, but could also be curved or otherwise configured to match the shape of a surface of the implant to be seated thereagainst.

Accordingly, upper portion 18b will deform less than lower portion 18a, and lower portion 18a will undergo more strain and deformation from the loading of elongate member 20 as it is secured in receiver 14 in contact with load transfer member 18.

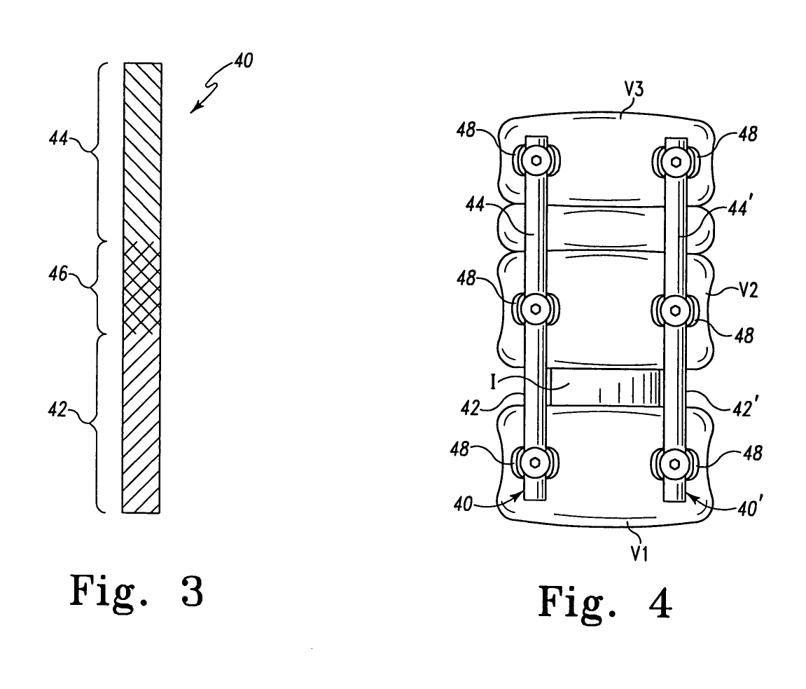

FIG. 3 represents another specific application for a medical device component including an elongated stabilization element 40 in the form of a spinal rod 40 having a first portion 42, a second portion 44, and a third portion 46 extending between the first and second portions 42, 44. Stabilization element 40 is a unitary structural component having a stiffness that varies along its length by varying the material properties in the various portions therealong. Stabilization element 40 can have a circular cross-sectional shape or any suitable non-circular cross-sectional shape. In addition, stabilization element 40 can include different cross-sectional shapes along its length. Stabilization element can be isotopic along all or a portion of its length and/or anisotropic along all or a portion of its length.

In one specific embodiment, stabilization element 40 is fabricated from a first material providing a first performance characteristic, such as a high modulus alloy Ti-6Al-4V, in first portion 42, and a second material having a second performance characteristic, such as a low modulus alloy Ti-15Mo, in second portion 44. Third portion 46 can provide a bonding layer that mixes these materials in a transition zone between the first and second portions 42, 44. Other embodiments contemplate that no transition portion or regions are provided. Still other embodiments contemplate more than two portions with each portion comprising a distinct material from the material of one or more of the other portions.

In yet another embodiment, transition region 46 can be comprised of a resorbable metal material such that the material in region 46 resorbs over time. The time for resorption can correspond to, for example, the time for fusion of one or more vertebral levels along which stabilization element 40 is attached. Once fusion of the one or more vertebral levels has been attained, stabilization element 40 has no stiffness since it separates into two or more portions.

One application for stabilization element 40 contemplates a spinal stabilization procedure where stabilization element 40 is secured along the spinal column with anchors 48 as shown in FIG. 4, for example. The stiffer first portion 42 can be engaged between first and second vertebrae V1, V2 where no or very little motion between the vertebrae is desired. One or more interbody implants I can be positioned in the disc space between vertebrae V1 and V2 for fusion of the vertebrae. Second portion 44, on the other hand, is less stiff and can be engaged between second and third vertebrae V2, V3 of another vertebral level where motion between the vertebrae is desired or permitted but where stabilization is desired during fusion of another vertebral level. Bi-lateral stabilization procedures with one or more other spinal stabilization elements 40′ like stabilization element 40 that also have first and second portions 42′, 44′ are also contemplated.

Anchors 48 can be secured to respective ones of the vertebrae V1, V2, V3 to engage stabilization element 40 along the vertebrae. Anchors 48 can be multi-axial, uni-axial, or uni-planar screws; fixed angle bone screws; variable angle bone screws; staples; wires or cables; suture anchor and sutures; interbody devices; intrabody devices; and combinations thereof, for example, that are suitable to secure stabilization element 40, 40′ to the respective vertebrae. In addition, stabilization along three or more levels or stabilization of a single vertebral level is contemplated.

In another embodiment, the stabilization element 40 can be secured along the spinal column with one or more of the anchors 10 discussed above.

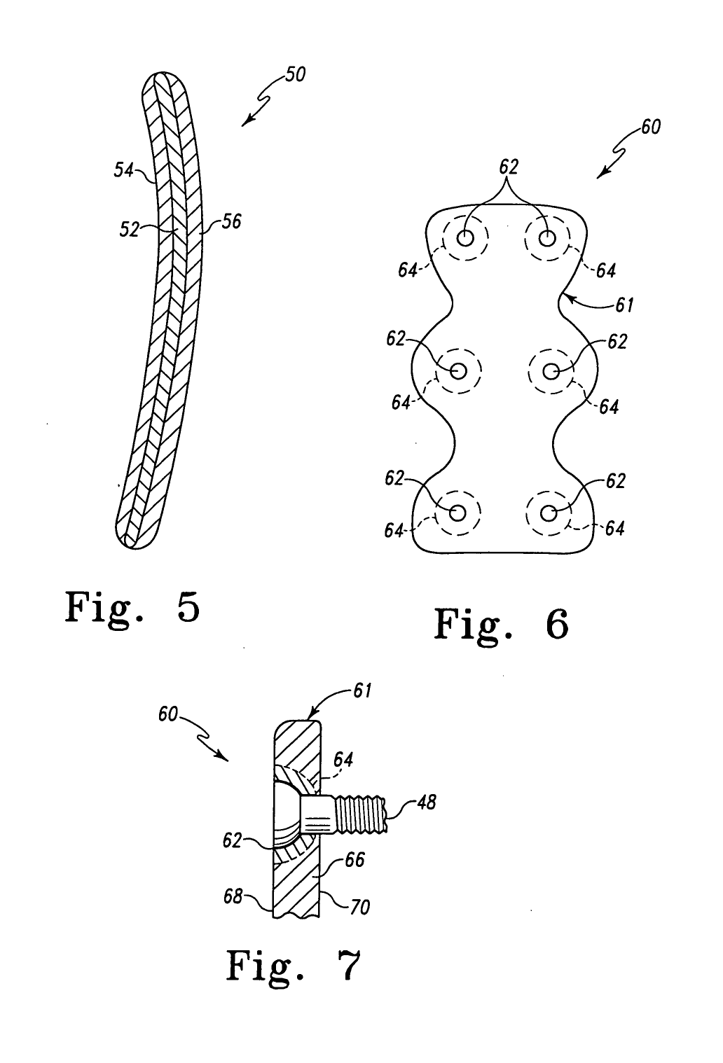

FIG. 5 represents another specific application of a component in the form of an elongated stabilization element 50 that can be a plate or rod, for example. Stabilization element 50 can be made, for example, to provide motion preserving performance characteristics with a first material along its length while retaining high strength performance characteristics with a second material. For example, stabilization element 50 can include layers formed by an inner portion 52 extending along its length and opposite outer portions 54, 56 extending along inner portion 52 along opposites sides thereof. Inner portion 52 can be made from a first material to provide a first performance characteristic, such as flexibility, to stabilization element 50. Outer portions 54, 56, on the other hand, can be made from a second material to provide high strength performance characteristics, such as fatigue resistant performance. In another example, inner portion 52 comprises a material with a lower modulus of elasticity and outer portions 54, 56 comprise a material with a high modulus of elasticity.

In another embodiment, the material layers are inverted so that a higher modulus material or fatigue-resisting material comprises the inner portion 52 and a lower modulus or flexible material comprises the outer portions 54, 56. Still other embodiments contemplate only two layers, or more than three layers. The lower or bone facing surfaces of stabilization element 50 can be curved along the longitudinal axis of stabilization element 50 as shown and/or curved transversely to the longitudinal axis of stabilization element 50.

FIGS. 6 and 7 show another specific application for a medical device component including elongated stabilization element 60 in the form of a plate 61 that is attachable to at least two vertebrae of a spinal column. Plate 61 includes an elongated body having a number of holes 62 extending between upper and lower surfaces 68, 70 thereof to receive bone anchors 48 to secure plate 61 to the spinal column. A first material can be provided on the plate in the portions 64 about the plate holes 62 that includes a performance characteristic that provides enhanced wear resistance of the plate at locations in contact with the bone engaging fasteners or anchors 48, while the remaining portion or portions 66 of the plate can be made from a material that provides a second performance characteristic such as flexibility.

While several specific applications have been shown and discussed above other specific applications are contemplated. For example, the component can also be a bone screw, a washer, a bolt, a set screw, a clamp, a staple, a crimp, or a connector, to name a few.

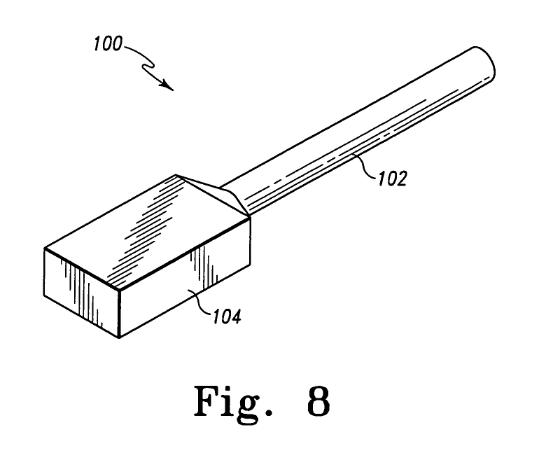

Also contemplated are medical devices in the form of surgical instruments where the instrument includes one or more portions fabricated so that one or more components or sub-components that include at least two constituent metals comprising different portions of the instrument. For example, with reference to FIG. 8, the surgical instrument 100 may include a first portion 102 in the form of an elongated shaft formed of a first metal or metal alloy, and a second portion 104 metallurgically joined to the first in the form of an end effector comprised of a second metal or metal alloy providing desirable performance characteristics to complete a surgical procedure. The end effector could includes means to manipulate tissue in the patient, and could be a cutting head, drill, reamer, forceps, distractor, holder, grasper, scraper, chisel, or an end of a cannula that is configured for expansion, cutting, or viewing, for example.

In specific embodiment, the first portion could be comprised of a metal or metal alloy providing flexibility to allow placement of the instrument into the body along non-linear insertion pathways, or providing stiffness to transmit forces to the end effector. The second portion could be comprises of a metal or metal material providing, for example, superior cutting capabilities, imaging properties;, flexibility, stiffness, wear resistance, hardness, or radiopacity. Examples of end effectors include those employed with cutting instruments, drills, reamers, distractors to separate bone portions, forceps, rongeurs, resection instruments, endoscopes, implant inserter instruments, bone tamps, retractors, and cannulae, for example

The present invention contemplates modifications as would occur to those skilled in the art without departing from the spirit of the present invention. In addition, the various procedures, techniques, and operations may be altered, rearranged, substituted, deleted, duplicated, or combined as would occur to those skilled in the art. All publications, patents, and patent applications cited in this specification are herein incorporated by reference as if each individual publication, patent, or patent application was specifically and individually indicated to be incorporated by reference and set forth in its entirety herein.

Any reference to a specific direction, for example, references to up, upper, down, lower, and the like, is to be understood for illustrative purposes only or to better identify or distinguish various components from one another. Any reference to a first or second vertebra or vertebral body is intended to distinguish between two vertebrae and is not intended to specifically identify the referenced vertebrae as adjacent vertebrae, the first and second cervical vertebrae or the first and second lumbar, thoracic, or sacral vertebrae. These references are not to be construed as limiting any manner to the medical devices and/or methods as described herein. Unless specifically identified to the contrary, all terms used herein are used to include their normal and customary terminology. Further, while various embodiments of medical devices having specific components and structures are described and illustrated herein, it is to be understood that any selected embodiment can include one or more of the specific components and/or structures described for another embodiment where possible.

Further, any theory of operation, proof, or finding stated herein is meant to further enhance understanding of the present invention and is not intended to make the scope of the present invention dependent upon such theory, proof, or finding.

What is claimed is:

1. An orthopedic device comprising:
an implant positionable in a patient in a surgical procedure;
a bone anchor assembly for engagement with at least one bony portion of the patient, said bone anchor assembly including:
a receiver engageable to the implant;
a bone engaging member extending from said receiver and including a first portion to engage the bony portion and a second portion adjacent the receiver; and
a load transfer member including a first portion adjacent said second portion of said bone engaging member and a second portion adjacent said implant, said first portion being comprised of a first material having a first performance characteristic and said second portion being comprised of a second material having a second performance characteristic that differs from said first performance characteristic, wherein said first and second materials are joined to provide an integral, unitary structure.

2. The device of claim 1, wherein said first portion deforms to conform to said second portion of said bone engaging member when said implant is secured in engagement against said second portion of said load transfer member and said second portion resists deformation when said implant is secured in engagement therewith.

3. The device of claim 1, wherein said first material is pure titanium and said second material is a titanium alloy having a higher yield strength than pure titanium.

4. The device of claim 1, wherein said first material is pure titanium and said second material is a titanium alloy having a higher stiffness than pure titanium.

5. The device of claim 1, wherein said first portion and said second portion of said load transfer member are metallurgically joined at an atomic level.

6. The device of claim 1, wherein said receiver includes a pair of arms forming said passage therebetween and further comprising an engaging member threadingly engageable to said arms to secure said implant in contact with said load transfer member.

7. The device of claim 1, wherein:
said first portion of said load transfer member includes a lower surface defining a concavely curved recess; and
said second portion of said bone engaging member includes an enlarged head received in said concavely curved recess.

8. The device of claim 7, wherein said head includes ridges for biting into said first portion of said load transfer member when said load transfer member is engaged thereagainst.

9. The device of claim 7, wherein said second portion of said load transfer member includes a seating surface opposite said recess for positioning in contact with said implant.

10. The device of claim 9, wherein said seating surface is flat and said implant is an elongated spinal rod.

11. The device of claim 1, wherein said receiver includes a passage for receiving said implant and said receiver includes an opening in communication with said passage, said bone engaging member extending through said opening and said second portion of said bone engaging member being pivotally mounted in said receiver, wherein said load transfer member locks said bone engaging member in position in said receiver when secured in engagement thereagainst.

12. An orthopedic device, comprising:
a body including at least a first portion and a second portion, wherein said first portion and said second portion provide said body with an integral, unitary structure and said first portion consists essentially of a first material having a first performance characteristic and said second portion consists essentially of a second material having a second performance characteristic that differs from said first performance characteristic; and
a system for securing said body to the spinal column.

13. The device of claim 12, wherein said body further includes a third portion between said first and second portions, said third portion including said first material and said second material.

14. The device of claim 12, wherein said body is elongated and in the form of a spinal rod and said system includes at least two anchors for engaging respective ones of first and second vertebral bodies and said spinal rod.

15. The device of claim 12, wherein said first portion includes a length sized to extend between vertebrae of a first vertebral level and said second portion includes a length sized to extend between vertebrae of a second vertebral level.

16. The device of claim 15, wherein said system includes a number of anchors to secure said body to vertebrae of the first and second vertebral levels.

17. The device of claim 16, wherein said first performance characteristic includes said first portion of said body having sufficient stiffness to immobilize the first vertebral level when secured thereto and said second performance characteristic includes said second portion of said body having sufficient flexibility to permit movement of the second vertebral level when secured thereto.

18. The device of claim 12, wherein said first performance characteristic includes a hardness that is greater than a hardness provided by said second performance characteristic.

19. The device of claim 12, wherein said body is elongate and in the form of a spinal plate having a number of holes therethrough and said anchor system includes bone screws positionable in said holes.

20. The device of claim 19, wherein said first portion forms a middle layer extending along said body and said second portion forms outer layers positioned along opposite sides of and extending along said middle layer.

21. The device of claim 19, wherein said first portion extends around said holes in said plate.

22. An orthopedic device comprising:
an elongate body positionable along bony portions, said body including at least a first portion extending along at least a first part of a length of said body and a second portion extending along at least a second part of a length of said body, wherein said first and second portions form an integral, unitary structure with said body and said first portion is comprised of a first material having a first performance characteristic and said second portion is comprised of a second material having a second performance characteristic that differs from said first performance characteristic;
an articulating bone screw assembly for engagement with the bony portion, said bone screw assembly including:
a receiver for receiving said body;
a bone engaging member extending from said receiver to engage at least one of the bony portions; and
a load transfer member between said bone engaging member and said body, said load transfer member contacting said bone engaging member and said implant when said implant is engaged to said receiver.

23. The device of claim 22, wherein said receiver includes a passage extending therethrough and an opening in communication with said passage, said bone engaging member extending through said opening and including a head portion adjacent said passage, said load transfer member further being located in said passage between said head portion and said elongate body.

24. The device of claim 23, wherein said load transfer member includes a first portion for engaging said head portion of said bone engaging member and a second portion for contacting said elongate body, wherein said first portion is made from a first material and said second portion is made from a second material, said first material being deformable to securely engage said head portion when said elongate body is seated against said second portion.

25. The device of claim 24, wherein said first portion and said second portion of said load transfer member are metallurgically joined to form a unitary body structure.

26. The device of claim 22, wherein said first performance characteristic of said first material provides a stiffness to resist movement of a first vertebral level when said first portion is secured therealong and said second performance characteristic of said second material provides a flexibility to permit movement of a second vertebral level when said second portion is secured therealong.

27. An elongated spinal implant device comprising:
a component comprising a first layer composed of a first metal material and positioned between second and third layers composed of a different, second metal material, wherein one of said first and second metal materials has a first stiffness that is less than a second stiffness of the other of said first and second metal materials, said component having a length between opposite ends thereof sized to extend between and be secured to at least two adjacent vertebrae, wherein said first, second and third layers provide an integral, unitary structure.

28. The device of claim 27, wherein the metal material of said first layer is selected from the group consisting of: titanium, titanium-aluminum-vanadium alloy, and titanium alloy.

29. The device of claim 27, wherein the metal material of said second and third layers is selected from the group consisting of: titanium, titanium-aluminum-vanadium alloy, and titanium alloys.

30. The device of claim 27, wherein said first layer is metallurgically joined to said second and third layers.

31. The device of claim 27, wherein said second layer forms a concavely curved bottom surface positionable against the at least two adjacent vertebrae and said third layer forms a convexly curved top surface facing away from the at least two adjacent vertebrae.

32. The device of claim 31 further comprising at least one hole extending through said first, second and third layers and opening at said top and bottom surfaces.

33. The device of claim 32 further comprising an anchor positionable in said at least one hole to secure said component to at least one the adjacent vertebrae.

34. The device of claim 31 wherein said component is an anterior cervical plate having a length sized to extend between at least two vertebrae and further includes a pair of holes extending between said top and bottom surfaces at respective ends of said plate for receiving anchors to secure said plate to least two vertebrae.

35. A method of fabricating a spinal implant, comprising:
providing a first portion of a component composed of a first metal;
providing a second portion of the component composed of a second metal, the second metal having a performance characteristic that differs from a performance characteristic of the first metal; and
joining the first portion and the second portion into an integral, unitary structure for the component, the component having a length sized to extend along at least first and second vertebrae when positioned along the spinal column.

36. The method of claim 35, wherein the unitary implant component is an anterior cervical plate.

37. The method of claim 36, wherein the first portion is substantially surrounded by the second portion.

38. The method of claim 37, wherein the first portion extends around holes extending through the plate.

39. The method of claim 36, wherein the first portion is an intermediate layer extending along a length of the component and the second portion includes second and third layers extending along opposite sides of the intermediate layer.

40. The method of claim 35, wherein the performance characteristic is selected from the group consisting of: hardness; deformability; flexibility; fatigue resistance; elasticity; wear resistance and radiopacity.

41. The method of claim 36, wherein the component is a spinal rod.

42. The method of claim 41, wherein the first portion of the spinal rod includes a length sized to extend between vertebrae at a first vertebral level and the second portion of the spinal rod includes a length sized to extend between vertebrae at a second vertebral level.

43. The method of claim 35, wherein said first portion and said second portion are joined metallurgically at an atomic level.

44. A method of fabricating a spinal implant, comprising:
providing a first portion of a component composed of a first metal;
providing a second portion of the component composed of a second metal, the second metal having a performance characteristic that differs from a performance characteristic of the first metal; and
joining said first portion and said second portion into an integral unitary structure for the component, the component having a first surface defined by the first portion having a first shape for seating against a second spinal implant and a second surface defined by the second portion for seating against a third spinal implant.

45. The method of claim 44, wherein said first surface is planar and said second surface is concavely curved.

46. The method of claim 44, wherein said first portion and said second portion are joined metallurgically at an atomic level.

47. A surgical instrument comprising:
a body including at least a first portion and a second portion, wherein said first portion and said second portion provide said body with an integral, unitary structure and said first portion consists essentially of a first material having a first performance characteristic and said second portion consists essentially of a second material having a second performance characteristic that differs from said first performance characteristic, wherein at least one of said first and second portions is an end effector including means for manipulating tissue of the patient.

48. The instrument of claim 47, wherein the other of the first and second portions is an elongated body for positioning the end effector in a location in the patient.

49. The instrument of claim 47, wherein said first and second performance characteristics are selected from the group consisting of: hardness; deformability; flexibility; fatigue resistance; elasticity; wear resistance and radiopacity.

50. The instrument of claim 47, wherein said first portion and said second portion are metallurgically joined to form a unitary body structure.
